(19) United States
(12) Patent Application Publication
(10) Pub. No.: US 2002/0183776 A1
(43) Pub. Date: Dec. 5, 2002

(54) Body piercing needle

(76) Inventor: Martin A. Ellis, Worcestershire (GB)

Correspondence Address: MARSHALL, GERSTEIN & BORUN, 6300 SEARS TOWER, 233 SOUTH WACKER, CHICAGO, IL, 60606-6357, US

(21) Appl. No.: 10/133,876

(22) Filed: Apr. 26, 2002

(30) Foreign Application Priority Data
Apr. 27, 2001 (GB) 0110343.1

(51) Int. Cl. A61B 17/34

(52) U.S. Cl. 606/185

(57) ABSTRACT

A need for use in body piercing for the attachment of an ornament to the body, the needle comprising a shank having a solid elongate main part, one end part of the shank terminating in a point and the other end of the shank having a head part comprising a pressure applying part which extends transversely to said shank.

### BACKGROUND TO THE INVENTION

This invention relates to a needle for use in body piercing for the attachment of an ornament to the body and to a method of attaching an ornament to a body.

### SUMMARY OF THE INVENTION

An object of the present invention is to provide a new and improved needle for this purpose. Another object of the invention is to provide a new and improved method of attaching an ornament to a body.

According to one aspect of the present invention we provide a needle for use in body piercing for the attachment of an ornament to the body, the needle comprising a shank having a solid elongate main part, one end part of the shank terminating in a point and the other end of the shank having a head part comprising a pressure applying part having a surface which extends transversely to said shank.

The main part of the shank may be of constant cross section.

The main part of the shank may be cylindrical.

The pressure applying part may comprise a surface which is continuous or substantially continuous.

The surface may be planar.

The surface may be of generally elongate shape.

The surface may have a minimum width dimension of 2-5 times the diameter of the main part of the shank and a maximum length dimension of 6-20 times said diameter.

The surface may be of generally diamond shape.

The generally diamond shape may have rounded corners.

The head part may include a tubular part which is disposed to extend generally perpendicular to the pressure applying part.

The pressure applying part may extend outwardly beyond the periphery of the tubular part at least at two diametrically opposite positions.

The tubular part may receive therein and be attached to the other end of the shank.

The tubular part may have an external outer surface which receives an elongate tubular cover which extends longitudinally over the shank to a position beyond the point.

The external surface of the tubular part and the internal surface of the cover may be formed to provide an interference fit.

Said interference fit may increase as the cover is moved in the direction from the tip towards the pressure applying part.

Preferably said outer surface of the tubular part is generally frustoconical.

The needle may be at least 35 mm long.

The needle may have an external diameter lying in the range 0.030 inches to 0.080 inches.

The shank of the needle may be disposed within a bore provided in a canular made of synthetic plastics material.

The canular may be circumferentially continuous.

The canular may have an internal diameter which is no more than 20% greater than the diameter of the main part of the shank.

The canular may be substantially inelastic.

The canular may be an interference fit on the main part of the shank.

The canular may be such that elastic deformation thereof permits an increase in the internal diameter of the canular by an amount selected from the group comprising 20%, 15%, 10%, 5%.

The canular may be adapted to extend from the head part to a position at or adjacent the inwardly disposed end of the point.

The part of the canular which is adjacent the point may have an external surface which is tapered so as to be of a reduced diameter externally adjacent the point and increase in diameter towards the main part of the canular.

The main part of the shank or the main part and the point may be provided with a lubricant such as silicon.

The bore and/or the external surface of the canular may be provided with a lubricant such as silicon.

The canular may have a wall thickness lying in the range 0.0055 inches to 0.0085 inches and preferably in the range 0.0065 inches to 0.0075 inches.

According to second aspect of the invention we provide a method of attaching an article to a body comprising the steps of piercing the body with a needle according to any one of the preceding claims and having a canular disposed on the shank to dispose the canular within the body, with drawing the needle shank from within the canular, whilst leaving the canular in the body disposing an end part of an article in an end part of the canular and withdrawing the canular and thereby introducing the part of the article into the body.

The end part of the article may be an interference fit in the canular.

The article may be manoeuvred in the body to a desired position.

An additional component or components may be attached to the article to retain the article in the body.

The part of the article may be an end part.

The body may comprise the flesh of a human being such as part of the ear.

### BRIEF DESCRIPTION OF THE DRAWINGS

One embodiment of the invention will now be described in more detail with reference to the accompanying drawings wherein:

FIG. 1 is a perspective view of a body piercing needle and cover embodying the invention,

FIG. 2 is a cross-section through the needle of FIG. 1.

FIG. 3 diagrammatically illustrates one stage in the attachment of an ornament of the body part, and

FIG. 4 diagrammatically illustrates an ornament attached to the body part.

### DETAILED DESCRIPTION OF THE PREFERRED EMBODIMENT AND BEST MODE OF THE INVENTION

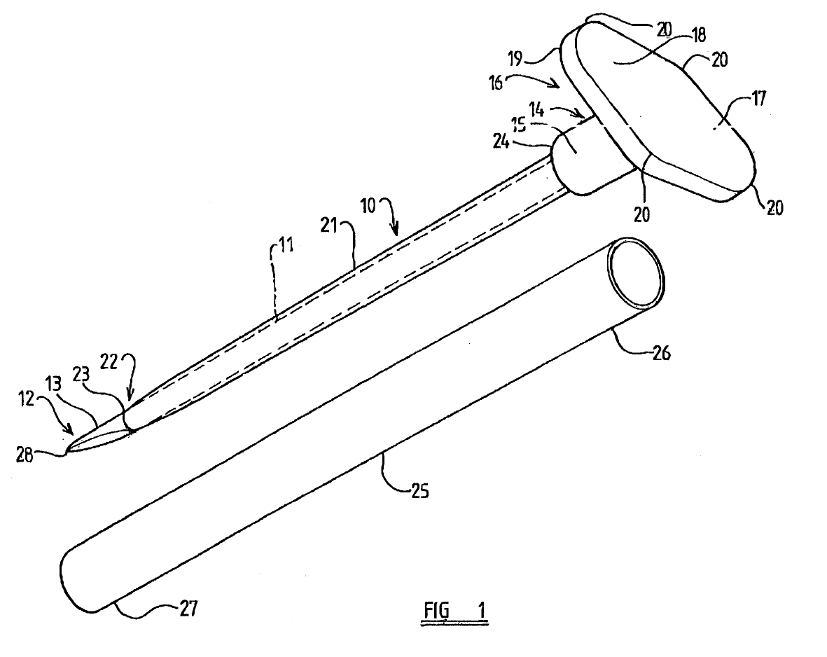

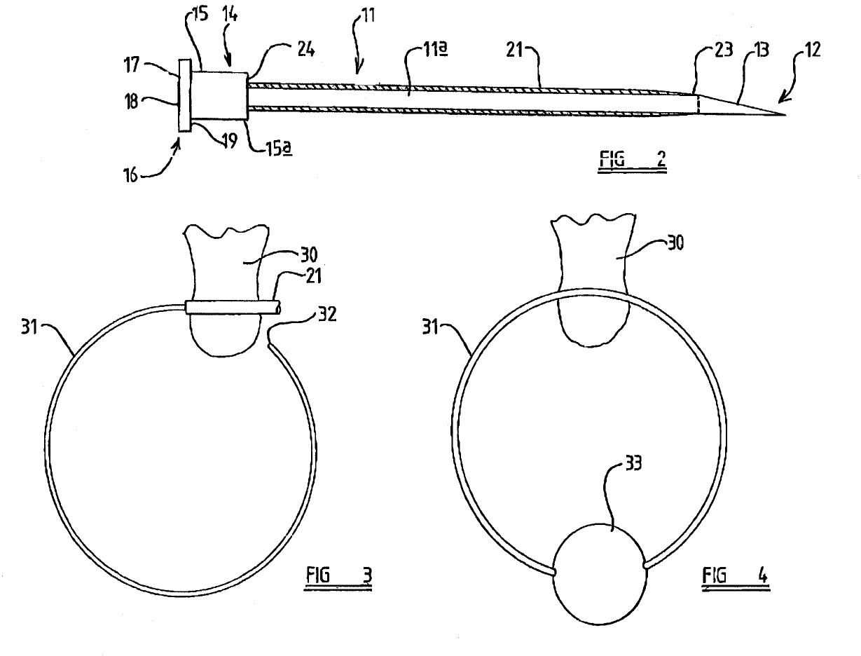

Referring to FIGS. 1 and 2, a needle for use in body piercing for the attachment of an ornament to the body is indicated generally at 10 and comprises a shank 11 having a solid elongate shank main part 11a. The main part 11a of the shank is of constant cross section throughout its length and is preferably cylindrical in shape.

The diameter of the cylinder may lie, for example, in the range 0.030 inches 0.080 inches. One end of 12 of the shank 11 terminates in a point 13 of conventional body piercing configuration whilst the other end 14 of the shank 11 is received within a bore of a tubular part 15 of a head part 16. No discontinuity consists between the main part 11 and the point 13. The head part 16 is attached to the shank 11 received therein in any desired suitable manner. The needle is at least 35 mm long.

The head part 16 also comprises a pressure applying part 17 which extends transversely to the shank 11. In the example illustrated the pressure applying part 17 has a planar surface 18 facing away from the point 13 and perpendicular to the shank 11. The pressure applying part 17 also has a planar under surface 19 facing towards the point 13. The surface 18 is of generally diamond shape having rounded corners as indicated at 20. If desired the surface 18 may have one or more apertures or depressions therein so long as it is adapted to enable body piercing pressure to be applied to the needle. Furthermore, if desired, the surface 18 may be other than planar for example it may be part tubular or part spherical in configuration again of such radius of curvature as to enable body piercing pressure to be applied to the needle. It is preferred that the pressure applying part is elongate and preferably has a minimum width dimension of 2-5 times the diameter of the main part 11a of the shank and a maximum length dimension of 6-20 times this diameter.

A canular 21 is received over the shank 11 of the needle. Canular 21 is made as a substantially inelastic plastic tube which is circumferentially continuous and is of generally cylindrical internal and external configuration but having an end part 22 which is tapered so as to produce a tip 23, adjacent to the inner end of the point 13, which is of reduced cross-section, as best shown in FIG. 2.

The canular 21 is preferably an interference fit on the main part 11a to minimise the risk of the canular deforming by buckling on insertion to provide an increased diameter and thereby greater discomfort to a patient. At most it is preferred that the internal diameter of the canular is no more than about 20% greater than the diameter of the needle. The wall thickness of the canular may lie in the range 0.0055 inches to 00.0085 inches and preferably lies in the range 0.0065 inches to 0.0075 inches. The main part 11a of the shank is unprovided, at least adjacent the tip 23, and in the present example throughout its length with a groove or any other abutment or discontinuity. As a result, when the needle is used to pierce a person's body there is no discernible step between the tip of the point 13 and the main part of the shank or between the needle and the canular. The canular 21 extends from the free end of the tip 23 towards the head 17 so as to abut an end surface 24 of the tubular part 15.

The canular is preferably at least 30 mm long.

A tubular cover 25, also of plastic, is provided for the needle and has an end part 26 which is formed to provide an interference fit with an external surface 15a of the tubular part 15. The arrangement illustrated has the external surface 15a configured to provided a shallow frusto-conical shape so as to provide an interference fit vith an internal surface 26a of the end part 26 of the cover 25. The cover 25 is of such a length that the end 27 thereof remote from the end 26 extends beyond the tip 28 of the point 13 thereby providing protection for the tip.

The main part of the shank 11 may be provided with a lubricant such as silicon. The internal and/or the external surface of the canular may be provided with a lubricant such as silicon.

In use, the needle, with the canular 21 thereon, is pushed through a part 30 of a patient's body such as, in particular, an ear of the patient. This is facilitated by the provision of the surface 18 which enables an operative comfortably to apply body piercing pressure to the needle in view of the relatively large size and the shape of the surface 18. The tubular portion of the read part 16 is intended to be grasped between the thumb and first finger of a right handed user whilst pressure is applied to the surface 18 by the third finger. The tubular portion facilitates grasping of the needle by the operative. When the canular 21 is correctly positioned through the body part 30 the needle shank is withdrawn. Because of the constant cross section of the main part 11a and the absence of the discontinuity between the main part 11a and the point 13 discomfort to a patient is minimised on piercing of the body of a patient with the needle. Moreover, the close fit on the needle and the minimal wall thickness of the canular further minimises a patient's discomfort on insertion of the device.

Since the canular is preferably at least 30 mm long and a patient's ear is typically 5-10 mm thick the needle and the canular thereon can project from both sides of the ear by a significant amount. Essentially a similar projecting effect is obtained with other parts of a patient's body which might be pierced.

An ornament, in the present example a ring 31, then has an end part inserted within the canular 21. The end part of the ornament is made an interference fit in the canular. To this end and also to prevent or minimise the above mentioned buckling the canular is made of relatively rigid synthetic plastics material of minimal elasticity. For example the elastic deformation is limited to that necessary to increase the internal diameter of the canular by 20% as a result of elastic deformation but may be 15, 10 or 5%. The canular 21 is then withdrawn thereby drawing the end part of the ring through the body part 30. The ornament is then manoeuvred so as to be positioned, generally as shown in FIG. 4, and the end parts 32 of the ornament are then disposed in recesses provided in, for example, a generally spherical ball 33 so that the ring 31 is closed and hence will be retained in the body part 30. If desired the ornament may be any desired article and it may be retained by one or more additional components.

By virtue of providing the pressure applying part 17 and arranging for it to extend beyond the shank of the needle significantly, in the present example by about two times the diameter of the shank in the minimum dimension direction of the surface 18 and ten times the diameter of the shank in the maximum dimension thereof a relatively large and convenient pressure applying surface 18 is provided so that the needle can be relatively easily grasped and inserted into the part 30 of the body.

In the present specification “comprise” means “includes or consists of” and “comprising” means “including or consisting of”.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A needle for use in body piercing for the attachment of an ornament to the body, the needle comprising a shank having a solid elongate main part, one end part of the shank terminating in a point and the other end of the shank having a head part comprising a pressure applying part having a surface which extends transversely to said shank.

2. A needle according to claim 1 wherein the main part of the shank is of constant cross section.

3. A needle according to claim 1 wherein the surface is of generally elongate shape.

4. A needle according to claim 1 wherein the surface has a minimum width dimension of 2-5 times the diameter of the main part of the shank and a maximum length dimension of 6-10 times said diameter.

5. A needle according to claim 1 wherein the head part includes a tubular part which is disposed to extend generally perpendicular to the pressure applying part.

6. A needle according to claim 5 wherein the tubular part has an external surface which receives an elongate tubular cover which extends longitudinally over the shank to a position beyond the point.

7. A needle according to claim 6 wherein the surface of the tubular part and the internal surface of the cover are formed to provide an interference fit.

8. A needle according to claim 1 wherein the needle is at least 35 mm long.

9. A needle according to claim 1 wherein the needle has an external diameter lying in the range 0.030 inches to 0.080 inches.

10. A needle according to claim 1 wherein the main part of the shank of the needle is disposed within a bore provided in a canular made of synthetic plastics material.

11. A needle according to claim 10 wherein the canular is circumferentially continuous.

12. A needle: according to claim 10 wherein the canular has an internal diameter which is no more than 20% greater than the diameter of the main part of the shank.

13. A needle according to claim 10 wherein the canular is substantially inelastic.

14. A needle according to claim 11 wherein the canular is an interference fit on the main part of the shank.

15. A needle according to claim 10 wherein the canular is such that elastic deformation thereof permits an increase in the internal diameter of the canular by an amount selected from the group comprising 20%, 15%, 10% and 5%.

16. A needle according to claim 10 wherein the part of the canular which is adjacent to the point has an external surface which is tapered so as to be of a reduced diameter externally adjacent the point and increase in diameter towards the main part of the canular.

17. A needle according to claim 1 wherein an element selected from the group comprising the main part of the shank, the main part and the point, the bore, the external surface, the canular, the bore and the external surface of the canular are provided with a lubricant such as silicon.

18. A needle according to claim 10 wherein the canular has a wall thickness lying in the range 0.0055 inches to 0.0085 inches.

19. A method of attaching an article to a body comprising the steps of piercing the body with a needle according to any one of the preceding claims and having a canular disposed on the shank to dispose the canular within the body, with drawing the needle shank from within the canular, whilst leaving the canular in the body disposing an end part of an article as an interference fit in an end part of the canular and withdrawing the canular and thereby introducing the part of the article into the body.

20. A method according to claim 19 wherein at least one additional component may be attached to the article retain the article in the body.
